(19) United States
(12) Patent Application Publication
(10) Pub. No.: US 2011/0313276 A1
(43) Pub. Date: Dec. 22, 2011

(54) NON-INVASIVE IN VITRO METHOD FOR QUANTIFYING LIVER LESIONS

(75) Inventors: Paul Cales, Avrille (FR); Christophe Aube, Angers (FR); Vincent Roullier, Andreze (FR)

(73) Assignees: CENTRE HOSPITALIER UNIVERSITAIRE D'ANGERS, Angers (FR); UNIVERSITE D'ANGERS, Angers (FR)

(21) Appl. No.: 13/129,999

(22) PCT Filed: Nov. 18, 2009
(86) PCT No.: PCT/IB2009/007719

§ 371 Date: Sep. 2, 2011

Related U.S. Application Data
(62) Division of application No. 61/115,677, filed on Nov. 18, 2008.

(51) Int. Cl. A61B 5/055; C12Q 1/52; G01N 33/68; G01N 33/98; C12Q 1/02; G01N 33/72; G01N 33/92; G01N 33/66; C12Q 1/56

(52) U.S. Cl. 600/410; 436/95; 435/16; 436/86; 435/13; 435/29; 436/66; 436/71; 436/94

(57) ABSTRACT

An in-vitro non-invasive method for quantifying the lesions of the liver of the patient with metabolic steatosis addressing a diagnostic target, i.e. fibrosis, steatosis and/or steato-hepatitis (NASH) and measuring at least one marker selected from the group consisting of biomarkers and possibly clinical markers and possibly scores, the biomarkers being selected from the group consisting of glycemia, AST (aspartate aminotransferase), ALT (alanine aminotransferase), AST/ALT, AST.ALT, ferritin, platelets, prothrombin index, hyaluronic acid, haemoglobin, triglycerides; the biomarker selected from weight, body mass index, sex and age, hip perimeter, abdominal perimeter and ratio thereof; the scores being selected from score of fibrosis, area of fibrosis, fractal dimension of fibrosis, score of steatosis, area of steatosis, fractal dimension of steatosis and combining the measures in a mathematical function combining the measures through a binary (or ordinal) logistic function or multiple linear regression including the markers in order to obtain an end value.

This invention relates to the field of hepatic diagnosis and more precisely to an in-vitro non-invasive method for quantifying liver lesions, especially due or related to liver impairment, liver steatosis, non-alcoholic fatty liver disease (NAFLD), or non-alcoholic steatohepatitis (NASH). By quantifying in the meaning of this invention is meant determining amount and/or architecture of hepatic lesions.

FLD (Fatty Liver Disease) describes a wide range of potentially reversible conditions involving the liver, wherein large vacuoles of triglyceride fat accumulate in hepatocytes via the process of steatosis (i.e. the abnormal retention of lipids within a cell).

FLD is commonly associated with alcohol or metabolic syndromes (diabetes, hypertension, dyslipidemia, abetalipoproteinemia, glycogen storage diseases, Weber-Christian disease, Wolman disease, acute fatty liver of pregnancy, lipodystrophy). However, it can also be due to nutritional causes (malnutrition, total parenteral nutrition, severe weight loss, refeeding syndrome, jejuno-ileal bypass, gastric bypass, jejunal diverticulosis with bacterial overgrowth), as well as various drugs and toxins (amiodarone, methotrexate, diltiazem, highly active antiretroviral therapy, glucocorticoids, tamoxifen, environmental hepatotoxins) and other diseases such as inflammatory bowel disease or HIV.

Whether it is AFLD (Alcoholic Fatty Liver Disease) or NAFLD (Non-Alcoholic Fatty Liver Disease), FLD encompasses a morphological spectrum consisting from the mildest type “liver steatosis” (fatty liver), called NAFL, to the potentially more serious type “steatohepatitis”, called NASH, which is associated with liver-damaging inflammation and, sometimes, the formation of fibrous tissue. In fact, steatohepatitis has the inherent propensity to progress towards the development of fibrosis then cirrhosis which can produce progressive, irreversible liver scarring or towards hepatocellular carcinoma (liver cancer).

Because these diseases can be potentially reversed if diagnosed early enough, or at least their consequences limited, it is crucial to be able to provide the medical field with tools that permitting such an early, rapid and precise diagnosis.

For a long time, the diagnosis of liver steatosis has usually been accomplished by measuring markers such as g-glutamyl-transpeptidase (GGT) and alanine aminotransferase (ALT) while at the same time performing a liver biopsy in order to confirm FLD and determine the grading and staging of the disease. Although biopsies can provide important information regarding the degree of liver damage, in particular the severity of necroinflammatory activity, fibrosis and steatosis, the procedure also presents several limitations, such as sampling error, invasiveness, cost, pain for patients which in turn brings forth a certain reluctance to undergo such a procedure; and finally complications may arise from such procedure, which in some cases can even lead to mortality. Ultrasonography is also used to diagnose liver steatosis. However, this method is subjective as it is based on echo intensity (echogenicity) and special patterns of echoes (texture). As a result, it is not sensitive enough and often inaccurate in patients with advanced fibrosis. Finally, it is generally admitted that around half of all FLD cases are detected by usual blood tests and around half by ultrasonography, resulting in around one quarter of missed diagnosis when both are used.

In recent years, the use of non-invasive biomarkers has gained importance in the field of hepatic diagnosis. Indeed, several tests using non-invasive biomarkers have already been developed and proposed for the diagnosis of fibrosis (see for example WO 2005/116901). The test relates to a method of diagnosing the presence and/or severity of a hepatic pathology and/or of monitoring the effectiveness of a curative treatment against a hepatic pathology in an individual, by establishing at least one non-invasive diagnostic score, in particular a diagnostic score for portal and septal fibrosis and/or an estimate for amount of fibrosis (the area of fibrosis) and/or an estimate for the architecture of fibrosis (fractal dimension).

Recently, Poynard et al. (WO 2006/082522) has demonstrated that a single or a panel of biomarkers can be used as an alternative to liver biopsy for the diagnosis of steatosis, whether induced by alcohol, viral hepatitis or NAFLD, the most common causes of steatosis. In particular, this document provides for a new panel of biomarkers known as a SteatoTest (ST) with predictive values for the diagnosis of steatosis due to alcohol, NAFLD and hepatitis C and B. Serum GGT and ALT were considered as the standard biochemical markers. According to the French National Agency for Health (HAS), updated in December 2008, and current international opinion, the performance of this kind of test may be insufficient, especially due to the reference based on a subjective grading of liver steatosis with a poor inter-observer reproducibility. In addition, this grading is a semiquantitative variable which implies an imprecise and limited reflect of the original pattern.

Watkins et al. (WO 2008/021192) has provided methods for assessing the level of triglycerides in the liver of a subject. Such methods comprise determining the amount of lipid metabolites in a sample collected from a body fluid of the subject and comparing it with a reference value representing the normal level of the lipid metabolites, or correlating the amount with the presence of a liver disorder. The methods are said to be used, for example, for the diagnosis and monitoring liver disorders such as steatosis, NAFLD and NASH (Non-Alcoholic Steato-Hepatitis). Thus, in order to predict steatosis, Watkins only uses one type of biomarkers (i.e. metabolic lipids) in a random body fluid. However, using this type of biomarkers has several limitations. First, it has been shown that serum triglyceride level was not always an independent marker of liver steatosis. Second, pathological processes are multiple step processes and a targeted strategy for biomarkers can miss one or several steps that are relevant for an accurate diagnosis. Finally, a body fluid is a result of mixing sources from the whole metabolism that can introduces some biases resulting from changes due to other organ than the target organ for diagnosis. Finally triglyceride content (biochemical measurement) is a different diagnostic target compared to liver steatosis (histological lesion, i.e. abnormal image)

The aim of the present invention is to provide new non-invasive methods using mixed biomarkers from different sources, which circumvent the above-mentioned limitations and are more precise and reliable than the methods cited in the prior art, as shown by their high diagnostic performance. The methods of the invention considerably reduce the need of biopsies, as they catch more information than in the prior art about the lesions evaluated, they ensure reproducibility and performance, while attenuating causes of false results (generally, sources of false results are antagonized in a score including several markers provided that some precautions are included).

The Applicant has now observed that, in the liver, the three lesions, fibrosis, steatosis and inflammation (NASH) are deeply interconnected. For example, the grade of liver steatosis is a predictive factor of NASH in patients with NAFLD; also, significant fibrosis is associated with the development of steatosis and NASH in NAFLD.

The Applicant also observed that, as a hepatic disease is due to the cause, leading to lesions, said lesions inducing symptoms, and ending up into complications, the most reliable information for the patient was the degree of his/her hepatic lesions.

The Applicant hereby shows that the lesions are related: there is a relationship between lesions.

Fibrosis may be quantified by determination of score (reflecting stage), area of fibrosis (reflecting amount) or fractal dimension (reflecting architecture).
Steatosis may be quantified by determination of score, area of fibrosis or fractal dimension.
NASH may be evaluated by determination of score(s).

This invention thus relates to an in vitro method for quantifying the lesions of a patient, preferably with NAFLD, comprising addressing a diagnostic target, i.e. quantifying fibrosis, steatosis and/or Nash by
carrying out a score, an area or a fractal dimension of fibrosis in said patient and/or
carrying out a score, an area or a fractal dimension of steatosis in said patient and/or
carrying out a score of steato-hepatitis (NASH) in said patient
   through
measuring at least one, preferably 2,3,4,5,6,7,8,9 marker(s) selected from the group consisting of biomarkers and possibly clinical markers and possibly scores, the biomarkers being selected from the group consisting of glycemia, AST (aspartate aminotransferase), ALT (alanine aminotransferase), AST/ALT, AST.ALT, ferritin, platelets, prothrombin index, hyaluronic acid, haemoglobin, triglycerides, the clinical biomarker being selected from the group consisting of weight, body mass index, sex and age and the scores being selected from the group consisting of score of fibrosis, area of fibrosis, fractal dimension of fibrosis, score of steatosis, area of steatosis, fractal dimension of steatosis;
and combining said measures in a mathematical function including said markers in order to obtain an end value.
   In the last step, combining is obtained by implementing the statistical method involving a mathematical function called binary (or ordinal) logistic regression or multiple linear regression with the following procedure:
first, the independent variables are tested by univariate analysis;
second, the independent variables that were significant in univariate analysis are tested in multivariable analysis by binary logistic (or ordinal regression or multiple linear regression with ascending or descending step by stepwise selection; the selection of variables can be performed in two ways
   The conventional one where the full sample of patient population is used. This selection procedure could also include score(s) of other lesions than the diagnostic target.
   The bootstrap method where the mathematical function is applied to different samples with less patients sorted by chance, usually 1000 samples. The selected independent variables are those with an absolute majority occurrence, i.e. at least 500 times among 1000 samples. This method also included only variables without significant colinearity (this means variables that are strongly linked). This procedure did not include score(s) of other lesions than the diagnostic target. Thus, this step provided a minimum number of predictive variables compared to the previous step.
the logistic regression produces the formula for each score in the form:

score=a0+a1x1+a2x2+ . . .

wherein the coefficients ai are constants and the variables xi are the independent variables; this score corresponds to the logit of p where p is the probability of existence of the diagnostic target. This probability p is calculated with the following formula:

p=exp(a₀+a₁x₁+a₂x₂+ . . . )/(1+exp(a₀+a₁x₁+a₂x₂+ . . . ))

or

p=1/(1+exp(−a₀−a₁x₁−a₂x₂− . . . ))

wherein the coefficients ai and the variables xi correspond to those of the formula for the score.

We give below the value of the associated coefficient ai (called [beta] in the text below and often in the literature and B in the tables below), and the last two columns give the ai confidence interval, i.e. the confidence interval (called CI in the tables below) of the beta coefficients or corresponding odds-ratio (called exp([beta]) in the tables).

When the diagnostic target is quantitative (discrete mathematical variable) such as the measurement of area of steatosis, the function used is a multiple linear regression with area of steatosis=

score=a0+a1x1+a2x2+ . . .

In one embodiment, the target is fibrosis, and a score, area and/or fractal dimension of fibrosis is performed, and at least 5, preferably 5,6,7,8 markers, are measured, said markers being selected from the group consisting of glycemia, AST, ALT, ferritine, platelets, prothrombin index, hyaluronic acid, haemoglobin, triglycerides, weight, body mass index, sex and age, preferably glycemia, AST, ALT, ferritin, platelets, weight and age or glycemia, AST, ALT, prothrombin index, weight.

In another embodiment, the target is steatosis and a score, area and/or fractal dimension of steatosis is performed, and at least 5 markers are measured, said markers selected from the group consisting of glycemia, AST, ALT, AST/ALT, AST.ALT ferritine, platelets, prothrombin index, hyaluronic acid, haemoglobin, triglycerides, weight, body mass index, sex and age, and preferably but not mandatory, a score, area or fractal dimension of fibrosis.

In a further embodiment, the target is steato-hepatitis, and a NASH score is performed.

In another embodiment, the diagnostic target is fibrosis, and
a score is carried out wherein the biomarkers are selected from the group consisting of weight, age, glycemia, AST, ALT, ferritin and platelets or from the group consisting of weight, age, glycemia, AST, ALT, ferritin and prothrombin index; or
an area is carried out wherein the biomarkers are selected from the group consisting of hyaluronic acid, glycemia, AST, ALT, platelets, and prothrombin index; or
a fractal dimension is carried out wherein the biomarkers are selected from the group consisting of weight, Hyaluronic acid, glycemia, AST, age, and prothrombin index.

In another embodiment, the diagnostic target is steatosis and
a score is carried out wherein the biomarkers are selected from the group consisting of glycemia, AST/ALT, triglycerides, haemoglobin, age, and sex and optionally at least one fibrosis score; or from the group consisting of glycemia, AST, triglycerides, AST/ALT, haemoglobin, BMI, and weight and optionally fibrosis scores and/or fractal dimension of fibrosis score; or from the group consisting of BMI, glycemia, and triglycerides, or from the group consisting of BMI, glycemia, triglycerides and ferritin; or
an area is carried out wherein the biomarkers are selected from the group consisting of glycemia, AST, triglycerides, BMI, weight and optionally at least one fibrosis score; or from the group consisting of BMI, glycemia, triglycerides and ferritin; or
a fractal dimension is carried out wherein the biomarkers are selected from the group consisting of glycemia, AST, ALT, triglycerides, BMI, weight and optionally at least one fibrosis score; or from the group consisting of BMI, glycemia, triglycerides and ferritin.

In another embodiment, the diagnostic target is NASH, and the biomarkers are selected from the group consisting of AST, ferritin, and AST. ALAT and optionally fibrosis score and/or steatosis score and/or NASH score; or from the group consisting of BMI, AST, and ferritin; or ferritin alone.

### Score#1

According to a first embodiment, the diagnostic target is significant fibrosis determined by Metavir scoring system implemented by Bedossa et al., and the method of the invention is performed by measuring the level of at least one, preferably 2,3,4,5,6,7, more preferably all markers selected from the group consisting of glycemia, AST, ALT, AST/ALT, ferritin, platelets and measuring the clinical markers weight and age, combining said measures through a mathematical function as described above with the conventional method.

| DIAGNOSTIC TARGET | | INDEPENDENT VARIABLES | SCORE |
|---|---|---|---|
| SIGNIFICANT | METAVIR | WEIGHT, AGE, GLYCEMIA, | #1A |
| FIBROSIS | F ≧ 2 | AST, ALT, FERRITIN, | |
| | | PLATELETS | |

Preferably, the coefficients of the mathematical function are as described in the examples.

When selected with the boostrap method, the variables are unchanged for this score.

| DIAGNOSTIC TARGET | | INDEPENDENT VARIABLES | SCORE |
|---|---|---|---|
| SIGNIFICANT | METAVIR | WEIGHT, AGE, GLYCEMIA, | #1B |
| FIBROSIS | F ≧ 2 | AST, ALT, FERRITIN, | |
| | | PLATELETS | |

Preferably, the coefficients of the mathematical function are as described in the examples.

### Score#2

According to a second embodiment, the diagnostic target is significant fibrosis determined by the NASH-CRN system implemented by Kleiner et al., and the method of the invention is performed by measuring the level of at least one, preferably 2,3,4, more preferably all markers selected from the group consisting of glycemia, AST, ALT, ferritin, prothrombin index and measuring the clinical markers age, weight and combining said measures through a mathematical function as described above with the conventional method.

| DIAGNOSTIC TARGET | | INDEPENDENT VARIABLES | SCORE |
|---|---|---|---|
| SIGNIFICANT | NASH-CRN | WEIGHT, AGE, GLYCEMIA, | #2A |
| FIBROSIS | F ≧ 2 | AST, ALT, FERRITIN, | |
| | | PROTHROMBIN INDEX | |

Preferably, the coefficients of the mathematical function are as described in the examples.

When selected with the boostrap method, the variables are less numerous for this score.

| | | | |
|---|---|---|---|
| SIGNIFICANT | NASH-CRN | WEIGHT, GLYCEMIA, AST, | #2B |
| FIBROSIS | F ≧ 2 | ALT, PROTHROMBIN INDEX | |

Preferably, the coefficients of the mathematical function are as described in the examples.

### Score#3

According to a third embodiment, the diagnostic target is area of fibrosis, and the method of the invention is performed by measuring the level of at least one, preferably 2, 3, 4, 5, 6, more preferably all markers selected from the group consisting of hyaluronic acid, glycemia, AST, ALT, platelets, prothrombin index and combining said measures through a mathematical function as described above.

| DIAGNOSTIC TARGET | INDEPENDENT VARIABLES | SCORE |
|---|---|---|
| AREA OF FIBROSIS | HYALURONIC ACID, | #3A |
| | GLYCEMIA, AST, ALT, | |
| | PLATELETS, | |
| | PROTHROMBIN INDEX | |

Preferably, the coefficients of the mathematical function are as described in the examples.

When selected with the boostrap method, the variables are unchanged for this score.

| | | |
|---|---|---|
| AREA OF FIBROSIS | HYALURONIC ACID, | #3B |
| | GLYCEMIA, AST, ALT, | |
| | PLATELETS, | |
| | PROTHROMBIN INDEX | |

Preferably, the coefficients of the mathematical function are as described in the examples.

### Score#4

According to a fourth embodiment, the diagnostic target is fractal dimension of fibrosis, and the method of the invention is performed by measuring the level of at least one, preferably 2, 3, 4, 5, more preferably all markers selected from the group consisting of hyaluronic acid, glycemia, AST, prothrombin index and the clinical marker weight, age, combining said levels in a mathematical function, obtained as described above.

| DIAGNOSTIC TARGET | INDEPENDENT VARIABLES | SCORE |
|---|---|---|
| FRACTAL DIMENSION | HYALURONIC ACID, | #4A |
| OF FIBROSIS | GLYCEMIA, AST, AGE, | |
| | PROTHROMBIN INDEX | |

Preferably, the coefficients of the mathematical function are as described in the examples.

When selected with the boostrap method, the variables are the following:

| | | |
|---|---|---|
| FRACTAL DIMENSION | HYALURONIC ACID, | #4B |
| OF FIBROSIS | GLYCEMIA, AST/ALT, | |
| | WEIGHT, PLATELETS | |

Preferably, the coefficients of the mathematical function are as described in the examples.

### Score#5

According to a fifth embodiment, the diagnostic target is significant steatosis determined according to a threshold of area of steatosis (3%), and the method of the invention is performed by measuring the level of at least one, preferably 2, 3, 4, 5, 6, more preferably all markers selected from the group consisting of glycemia, AST/ALT, triglycerides, haemoglobin, age and sex, and optionally a score as obtained from the first embodiment (score #1A) or the second embodiment (score#2A) of the present invention, as described above, and combining said levels in a mathematical function, obtained as described above.

| DIAGNOSTIC TARGET | | INDEPENDENT VARIABLES | SCORE |
|---|---|---|---|
| Significant | rAOS ≧ 3% | GLYCEMIA, AST/ALT, | #5A |
| steatosis | | TRIGLYCERIDES, | |
| | | HAEMOGLOBIN, AGE, | |
| | | SEX + optionally SCORE | |
| | | 1A AND/OR SCORE 2A | |

Preferably, the coefficients of the mathematical function are as described in the examples.

When selected with the boostrap method, the variables are the following:

| | | | |
|---|---|---|---|
| SIGNIFICANT | rAOS ≧ 3% | BMI, GLYCEMIA, | #5B |
| STEATOSIS | | TRIGLYCERIDES | |

Preferably, the coefficients of the mathematical function are as described in the examples.

### Score #6

In the sixth embodiment, the diagnostic target is significant steatosis determined by NASH-CRN GRADE≧1, and the method of the invention is performed by measuring the level of the biomarkers selected from glycemia, AST, triglycerides, AST/ALT, haemoglobin, the clinical markers are selected from the group consisting of BMI and weight, and optionally, one or more of the score 1, 2 and/or 4 and combining said levels in a mathematical function, obtained as described above.

| DIAGNOSTIC TARGET | | INDEPENDENT VARIABLES | SCORE |
|---|---|---|---|
| SIGNIFICANT | NASH-CRN | GLYCEMIA, AST, | #6A |
| STEATOSIS | GRADE ≧ 1 | TRIGLYCERIDES, AST/ALT, | |
| | | HAEMOGLOBIN, BMI, | |
| | | WEIGHT + SCORES 1A, 2A | |
| | | AND 4A | |

Preferably, the coefficients of the mathematical function are as described in the examples.

When selected with the boostrap method, the variables are the following:

| | | | |
|---|---|---|---|
| SIGNIFICANT | NASH-CRN | BMI, GLYCEMIA, | #6B |
| STEATOSIS | GRADE ≧ 1 | TRIGLYCERIDES, | |
| | | FERRITIN | |

Preferably, the coefficients of the mathematical function are as described in the examples.

### Score #7

In the seventh embodiment, the diagnostic target is area of steatosis, and the method of the invention is performed by measuring the level of at least one, preferably 2, 3, 4, 5, more preferably all markers selected from the group consisting of glycemia, AST, triglycerides, ferritin, BMI and weight, and optionally, one or more of the scores 1 and/or 2, combining said levels in a mathematical function, obtained as described above.

| DIAGNOSTIC | | |
|---|---|---|
| TARGET | INDEPENDENT VARIABLES | SCORE |
| AREA OF | GLYCEMIA, AST, TRIGLYCERIDES, | #7A |
| STEATOSIS | BMI, WEIGHT + OPTIONNALY | |
| | SCORES 1 AND 2 | |

Preferably, the coefficients of the mathematical function are as described in the examples.

When selected with the boostrap method, the variables are the following:

| | | |
|---|---|---|
| AREA OF STEATOSIS | BMI, GLYCEMIA, | #7B |
| (LOG) | TRIGLYCERIDES, | |
| | FERRITIN | |

Preferably, the coefficients of the mathematical function are as described in the examples.

### Score#8

In the eighth embodiment, the diagnostic target is fractal dimension of steatosis, and the method of the invention is performed by measuring the level of at least one, preferably 2, 3, 4, 5, more preferably all markers selected from the group consisting of glycemia, AST, ALT/ALAT, AST.ALT, ferritin, triglycerides, BMI, weight, and optionally scores 1A and 2A and combining said levels in a mathematical function, obtained as defined in embodiment 1 and 2 here above.

| | | |
|---|---|---|
| FRACTAL DIMENSION | GLYCEMIA, AST, ALT/ALAT, | #8 |
| OF STEATOSIS | AST.ALT, TRIGLYCERIDES, | |
| | BMI, WEIGHT + | |
| | SCORES 1A AND 2A | |

Preferably, the coefficients of the mathematical function are as described in the examples.

When selected with the boostrap method, the variables are the following:

| | | |
|---|---|---|
| FRACTAL DIMENSION | BMI, GLYCEMIA, | #8B |
| OF STEATOSIS | TRIGLYCERIDES, | |
| | FERRITIN | |

Preferably, the coefficients of the mathematical function are as described in the examples.

### Score #9

In the ninth embodiment, the diagnostic target is NASH according to three definitions based on NASH activity score (NAS) by Kleiner et al., and the method of the invention is performed by measuring the level of at least one, preferably 2, 3, 4, more preferably all markers selected from the group consisting of AST, ferritin, AST.ALT, BMI, and optionally scores 2A and/or 5A and/or 8A and combining said levels in a mathematical function, obtained as described above.

| | | |
|---|---|---|
| NASH | AST, FERRITIN, AST.ALAT + | #9A |
| | OPTIONALLY SCORES 2A, 5A | |
| | AND/OR 8A | |

Preferably, the coefficients of the mathematical function are as described in the examples.

When selected with the boostrap method, the variables are the following:

| | | |
|---|---|---|
| NAS ≧ 3 | BMI, AST, FERRITIN | #9B |
| NAS ≧ 5 | FERRITIN | #10B |
| NAS 0-2/3-4/5-7 | BMI, AST, FERRITIN | #11B |

Preferably, the coefficients of the mathematical function are as described in the examples.

In the context of the present invention, the following definitions are to be applied:
Fibrosis is defined as a pathological lesion made of scaring tissue including fibrillar proteins or glycoproteins (collagens, proteoglycans . . . ).
Fibrosis Score is defined as a combination of markers (usually linked by a mathematical function) for which the diagnostic target is fibrosis.
Steatosis is defined as accumulation of lipids, usually triglycerides, within vacuoles in cells. It mainly concerns liver and muscle in metabolic syndrome.
Area of steatosis (AOS) is defined as the liver surface occupied by the vacuoles of steatosis as seen on a liver specimen observed in two dimensions.
Significant area of steatosis (SAOS) is defined as a threshold clinically and statistically significant of AOS, in the setting of NAFLD, e.g. more than or equal to 3% of liver surface.
Kleiner grading/staging: this is a pathological classification devoted to NAFLD based on a morphological description in different classes either for steatosis (conventionally referred as grading) or fibrosis (conventionally referred as staging). This semi-quantitative (ordinal in statistics) system is the most recent and conventional histological classification. This system is also known as the NASH Clinical Research Network (NASH-CRN) system.
Metavir grading/staging: this is a pathological classification devoted to chronic viral hepatitis based on a morphological description in different classes either for necro-inflammatory activity (conventionally referred as grading) or fibrosis (conventionally referred as staging). This semi-quantitative (ordinal in statistics) system is the most used histological classification in viral hepatitis. It is also used in other causes of chronic liver diseases such as alcohol and NAFLD.
Level: the quantity of a particular biomarker measured in the blood or plasma of a patient, usually expressed as a concentration.

Target is Steatosis

The Applicant especially focused on the target “steatosis” and found that the inclusion of fibrosis score in an in vitro method for diagnosing a liver condition involving steatosis, might be of high interest because of the interaction between fibrosis and steatosis (see FIG. 2 showing that the relationship between AOS and fibrosis stages explains that the inclusion of non-invasive measurement of fibrosis stage increases the accuracy of non invasive measurement of AOS)

Thus, according to an embodiment, a method of the invention comprises measuring the level of at least one, preferably 2, 3, 4, 5, 6, 7, 8 biological marker (s) and at least one, preferably two, clinical marker (s) and optionally, but preferably, a Fibrosis Score, and combining said levels, measures and score in a suitable mathematical function.

According to a first embodiment, the at least one biological marker is selected from the group consisting of glycemia (GLY), aspartate aminotransferase (AST), alanine aminotransferase (ALT), AST/ALT, hyaluronic acid (HA), Haemoglobin (Hb) and triglycerides, preferably glycemia (GLY), aspartate aminotransferase (AST), alanine aminotransferase (ALT), AST/ALT, hyaluronic acid (HA), Haemoglobin (Hb) and triglycerides; the level of these blood markers may be easily dosed with methods already known in the art; preferably two clinical markers are selected from the group consisting of age, sex, BMI, weight; preferably, a score is performed.

According to a second embodiment, the Fibrosis Score of the present invention is determined through the method described in WO2005/116901, herein incorporated by reference.

According to a further embodiment, the Fibrosis Score is determined by measuring in a sample of said patient and combining in a logistic regression function at least three markers selected in the group consisting of α-2 macroglobulin (A2M), hyaluronic acid (HA or hyaluronate), apoliprotein A1 (ApoA1), N-terminal propeptide of type III procollagen (P3P), gamma-glutamyltranspeptidase (GGT), bilirubin, gamma-globulins (GLB), platelets (PLQ), prothrombin level (TP), aspartate amino-transferase (AST), alanine amino-transferase (ALT), urea, sodium (NA), glycemia (GLY), triglycerides (TG), albumin (ALB), alkaline phosphatases (PAL), YKL-40 (human cartilage glycoprotein 39), tissue inhibitor of matrix metalloproteinase 1 (TIMP-1), matrix metalloproteinase 2 (MMP-2), ferritin.

According to another embodiment, the Fibrosis Score is measured by combining the levels of at least three biological markers selected from the group consisting of glycemia (GLY), aspartate aminotransferase (AST), alanine amino-transferase (ALT), ferritine, hyaluronic acid (HA), triglycerides (TG), prothrombin index (PI) gamma-globulins (GLB), platelets (PLQ), weight, age and sex.

According to yet another embodiment, at least 2, preferably at least 3, more preferably at least 4, even more preferably at least 5 biological markers, and even most preferably at least 6 biological markers and optionally Fibrosis Score are measured and combined in the method of the present invention.

According to another embodiment, the method according to the invention further comprises measuring at least one clinical marker. Preferably the clinical marker is selected from the group consisting of the age (AGE), the body mass index (BMI), the body weight, the hip perimeter, the abdominal perimeter and a ratio thereof, such as for example hip perimeter/abdominal perimeter; more preferably two, clinical markers are measured.

According to yet another embodiment, the method according to the invention comprises measuring at least three biomarkers selected from the group consisting of glycemia (GLY), aspartate aminotransferase (AST), alanine aminotransferase (ALT), hyaluronic acid (HA), Hemoglobin (Hb) and triglycerides, and at least one, preferably two clinical markers selected from the group consisting of the age (AGE), the body mass index (BMI), the body weight, the hip perimeter, the abdominal perimeter and a ratio thereof, such as for example hip perimeter/abdominal perimeter and a Fibrosis score and combining said levels of biological markers, measured Fibrosis Score and measured clinical markers, through a suitable mathematical function, preferably a logistic function or a multiple regression function.

Steatosis by MRI

Another object of the present invention is a method for quantifying the area of liver steatosis (AOS) in a patient, comprising performing a multi-echo gradient-echo MRI called MFGRE on whole or part of the liver of the patient, measuring the liver fat content on the resulting MRI signal and comparing said liver fat content on the resulting MRI signal to the area of lipid vacuoles of the reference image.

(MFGRE) is a new method developed by the applicant and based on a multi-echo prototype MRI sequence. The principle of the sequence is an echo gradient in-phase and out-of-phase but with the acquisition of 16 echos, allowing the precise calculation of signal decay parameters: water signal (W), fat (F) signal, local field in homogeneity (F) and noise (N). Fat fraction is calculated using the following formula:

$$\frac{F}{W + F} \times 100 = \% {F}_{MGRE}$$

With this method, MFGRE is used as a non invasive marker of steatosis either per se or with equivalence in AOS by using a linear regression score. This marker can be used as part of a test in the same way as a blood or clinical marker.

A third object of the present invention is a method for quantifying an area of liver steatosis (AOS) in a patient, comprising:

a) measuring the fat content on a MRI signal of the liver of a patient;
b) measuring the level of at least one biological marker and/or Fibrosis Score, and/or at least one, preferably two, clinical marker(s);
c) combining the measured fat content index obtained in step (a) and the measured levels, score and/or clinical markers obtained in step (b) in a suitable mathematical function.

According to yet another embodiment, the method for quantifying an area of liver steatosis (AOS) measures at least 2, preferably at least 3, more preferably at least 4 and even more preferably at least 5 biological markers and most preferably at least 6 biological markers and optionally Fibrosis Score in step (b).

A fourth object of the present invention is a method for diagnosing the presence and/or the severity of a liver steatosis or fatty liver disease in a patient, comprising implementing a method of measuring the area of steatosis as described in the present invention. The method of the invention is of high interest to diagnose and quantify main liver lesions, especially NAFLD lesions.

Other objects, advantages and features of the present invention will become more apparent upon reading of the following non restrictive description of preferred embodiments thereof, given by way of examples with reference to the accompanying figures.

### BRIEF DESCRIPTION OF THE FIGURES

FIG. 1 Correlation between morphometric characteristics of liver fibrosis or steatosis and corresponding blood tests as a function of fibrosis stages or steatosis grades. Panel a: area of fibrosis, panel b: fractal dimension of fibrosis. Panel c: area of steatosis, panel d: fractal dimension of steatosis. The lines depict the linear regression.

FIG. 2 Relationship between the area and fractal dimension of steatosis as a function of steatosis grades. The lines depict the LOWESS regression curves. The Y axis was truncated to show more details.

FIG. 3 Relationship between blood tests for NAS (panel a) or NASH (panel b) (Y axis) and NAS (X axis). Box plots (median, interquartile range and extremes).

FIG. 4 Relationship between area and fractal dimension as a function of fibrosis stages or steatosis grades. Left panels (a and c): morphometric results. Right panels (b and d): blood tests. Upper panels (a and b): fibrosis; bottom panels (c and d): steatosis. The lines depict the LOWESS regression curves.

FIG. 5 Relationship between area or fractal dimension of fibrosis or steatosis (Y axis) as a function of fibrosis stages (X axis). Left panels (a and c): morphometric results. Right panels (b and d): blood tests. Upper panels (a and b): area; bottom panels (c and d): fractal dimension. Box plots (median, interquartile range and extremes).

FIG. 6 depicts the correlation between the AOS (X axis) and the MFGRE sequence by MRI (Y axis) in 23 patients: Rs=0.77 and Ric=0.85.

### EXAMPLES

### Patients and Methods

### Patients

Two tertiary centers, Angers and Rennes (France), prospectively recruited 245 patients with NAFLD between 2001 and 2006. Inclusion and exclusion criteria are described elsewhere (Cales P et al, J Hepatol 2009, 50:165-173). Nineteen patients were not included because their liver specimens were not available for rereading; thus, the core population included 226 patients. Additional 22 liver biopsies were unsuitable for image analysis.

### Clinical Data and Blood Tests

Fasting blood samples were taken at inclusion (date of liver biopsy±7 days). The usual clinical and blood variables were included, especially fasting glucose at inclusion, as were the following fibrosis markers: hyaluronic acid, α2-macroglobulin, apolipoprotein-A1, prothrombin index, platelets, aspartate and alanine aminotransferases (AST, ALT), γ-glutamyltranspeptidase, total bilirubin and urea. Methods and reagents were previously described (Cales P et al, Clin Biochem 2008, 41:10-18). One blood fibrosis score specific for NAFLD was calculated according to published score (Cales P et al, J Hepatol 2009, 50:165-173). The inter-laboratory reproducibility of blood markers was evaluated as excellent in a previous study (Cales P et al, Clin Biochem 2008, 41:10-18).

### Liver Biopsy

A percutaneous liver biopsy was performed generally within one week (maximum 3 months) of blood sampling. Specimen lengths were measured before paraffin-embedding. Then, 5 μm-thick sections were stained with hematoxylin-eosin-saffron or 0.1% picrosirius red solution and used for both optical and image analyses.

### Optical Analysis

Biopsy specimens were centrally re-examined by two liver pathologists; discordant cases were reviewed by a third pathologist to reach consensus. Steatosis, lobular inflammation and hepatocyte ballooning were separately graded according to the NASH Clinical Research Network (NASH-CRN) system (Kleiner DE et al, Hepatology 2005, 41:1313-1321). The addition of these 3 grades provided the NAFLD activity score (NAS). The diagnosis of NASH was evaluated according to three definitions based on NAS: ≧3, ≧5 and 0-2/3-4/5-7, respectively no, possible/borderline, definite NASH (Kleiner DE et al, Hepatology 2005, 41:1313-1321). Fibrosis was graded into 5 stages, called here F0 to F4, according to NASH-CRN staging specifically developed for NAFLD (Kleiner DE et al, Hepatology 2005, 41:1313-1321). The Metavir staging (Hepatology 1994, 20: 15-20) developed for viral hepatitis, but also used in alcoholic chronic liver diseases and in NAFLD, was also evaluated to stage porto-septal fibrosis in acinar zone 1.

### Image Analysis

Image acquisition—We used an Aperio digital slide scanner (Scanscope® CS System, Aperio Technologies, Vista Calif. 92081, USA) image processor that provided high quality 30,000×30,000 pixel images at a resolution of 0.5 μm/pixel (magnification ×20). A binary image (white and black) was obtained via an automatic thresholding technique using an algorithm developed in our laboratory. The entire specimen area was analyzed. Liver specimen artifacts (folds, dust) were manually removed.

Algorithm for steatosis—Some aspects of the algorithm have been previously described (Roullier V et al., Conf Proc IEEE Eng Med Biol Soc 2007; 2007:5575-5578) and techniques providing images and measurements of AOS have been recently developed (Boursier J et al., J Hepatol 2009, 50:S357).

Calculated data—AOF was measured on the complete liver sections, as previously described (Pilette C et al., J Hepatol 1998, 28:439-446) but with several improvements in image acquisition as described above. AOS (%) was calculated as the ratio: area of steatosis vesicles/complete liver surface, and relative AOS (rAOS, %) as the ratio: area of steatosis/non-fibrous liver area (i.e. complete liver surface minus AOF). Finally, the box counting method was used to evaluate the Kolmogorov FD of steatosis and fibrosis as detailed elsewhere (Moal F et al., Hepatology 2002, 36:840-849).

### Statistical Analysis

Quantitative variables were expressed as mean±SD, unless otherwise specified. The Lowess regression by weighted least squares was used to determine the average trend of relationships between variables (Borkowf C B et al. Stat Med 2003, 22:1477-1493). Independent predictors were determined by the bootstrapping method. This method was also determined to calculate the optimism bias.

Diagnostic performance was expressed by the area under the receiver operating characteristic (AUROC) or diagnostic accuracy. Diagnostic cut-offs were determined according to maximum Youden index and diagnostic accuracy. Stepwise multiple linear regression and binary logistic regression were used to determine independent variables. In the linear regression, all categorical variables were dichotomized and the performance of each model was expressed by the adjusted R² (aR²). Statistics software programs were SPSS version 11.5.1 (SPSS Inc., Chicago, Ill., USA) and SAS 9.1 (SAS Institute Inc., Cary, N.C., USA).

### Results

### General Characteristics of NAFLD Patients

They are detailed in table 1.

TABLE 1
Characteristics of 226 patients with NAFLD included.

| Variable | Value (% or mean±) |
|---|---|
| Demographic: | |
| Sex (% male) | 75.2 |
| Age (years) | 50.9 ± 10.8 |
| Body weight (kg) | 82.9 ± 15.8 |
| BMI (kg/m²) | 28.7 ± 4.9 |
| Liver specimen: | |
| Size^{a} (mm) by pathologist | 30.5 ± 11.9 |
| Size^{b} (mm) by computer | 23.4 ± 23.6 |
| Area^{b} (mm² ) | 11.7 ± 11.8 |
| Fibrosis: | |
| Fibrosis stage by Metavir (% | 44.2/29.2/9.3/ |
| Fibrosis stage by NASH-CRN (% | 26.1/29.7/21.6/ |
| Area of fibrosis (%) | 6.4 ± 5.4 |
| Fractal dimension | 1.14 ± 0.20 |
| Steatosis: | |
| Steatosis grade (% grade 0/1/2/3) | 45.3/21.5/21.1/ |
| Area of Steatosis (%) | 10.3 ± 9.7^{c} |
| Fractal dimension | 1.52 ± 0.17 |
| NAFLD activity score: 0-2, 3-4, 5-7 | 53.2/30.6/16.2 |
| NASH (%) | 46.8 |

^{a}Before paraffin embedding;
^{b}By computerized analysis of stained digital images;
^{c}Range: 0.2-42.5%;
^{d}NAS: interpretation: no, possible/borderline, definite NASH.

### Diagnosis of Fibrosis

### Significant Fibrosis

Diagnostic performance—The prevalence of significant fibrosis was significantly higher with NASH-CRN F≧2: 43.9% than with Metavir F≧2: 27.6% (p<10³ by McNemar test). We have compared a previous blood test targeted to Metavir F≧2, called FibroMeter (FM) and a new model of blood test targeted to NASH-CRN F≧2 (table 2). Tables 2 and 3 show very good performance (high AUROC) for the scores of the invention.

TABLE 2
Multivariate analyses showing variables independently
associated with different diagnostic targets.

| | | Independent | | |
|---|---|---|---|---|
| Diagnostic target | | variables | Score | Performance |
| Fibrosis: | | | | |
| Significant | Metavir | Weight, age, | #1A | AUROC = 0.940 |
| fibrosis | F ≧ 2 | glycemia, AST, | | |
| | | ALT, ferritin, | | |
| | | platelets | | |
| | NASH-CRN | Weight, age, | #2A | AUROC = 0.884 |
| | F ≧ 2 | glycemia, AST, | | |
| | | ALT, ferritin, | | |
| | | prothrombin | | |
| | | index | | |
| Area of fibrosis | | Hyaluronic acid, | #3A | aR2 = 0.520 |
| | | glycemia, AST, | | |
| | | ALT, platelets, | | |
| | | prothrombin index | | |
| Fractal dimension of | | Hyaluronic acid, | #4A | aR2 = 0.524 |
| fibrosis | | glycemia, AST, | | |
| | | age, prothrombin | | |
| | | index | | |
| Steatosis: | | | | |
| Significant | rAOS ≧ 3% | Glycemia, | #5A | AUROC = 0.847 |
| steatosis | | AST/ALT, | | |
| | rAOS ≧ 3% | triglycerides, | #5A | AUROC = 0.847 |
| | | haemoglobin, age, | | |
| | | sex + scores 1 | | |
| | | and 2 | | |
| | NASH-CRN | Glycemia, AST, | #6A | AUROC = 0.848 |
| | grade ≧ 1 | triglycerides, | | |
| | | AST/ALT, | | |
| | | haemoglobin, BMI, | | |
| | | weight + scores | | |
| | | 1, 2 and 4 | | |
| Area of steatosis | | Glycemia, AST, | #7A | aR2 = 0.365 |
| | | triglycerides, | | |
| | | BMI, weight + | | |
| | | scores 1 and | | |
| Fractal dimension of | | Glycemia, AST, | #8A | aR2 = 0.346 |
| steatosis | | ALT, triglycerides, | | |
| | | BMI, weight + | | |
| | | scores 1 and 2 | | |
| NASH: | | | | |
| NASH | | AST, ferritin, | #9A | AUROC = 0.895 |
| | | AST.ALAT + | | |
| | | scores 2, 5 | | |
| | | and 8 | | |

Diagnostic performance was expressed by either AUROC for score obtained by binary logistic regression or ₐR² for score obtained by multiple linear regression. In these models, the prediction was the main objective so that conditions of independent variables were less stringent than in an explanatory model.
AST: aspartate aminotransferase
ALT: alanine aminotransferase
BMI: body mass index

TABLE 2bis
Multivariate analyses showing variables independently
associated with different diagnostic targets.

| | | | | | 95% CI | 95% CI |
|---|---|---|---|---|---|---|
| | Diagnostic | | β | | inf | sup |
| Score | target | Variables | coeff | Exp (β) | Exp (β) | Exp (β) |
| | Significant | | | | | |
| | fibrosis | | | | | |
| | Metavir | | | | | |
| 1a | F ≧ 2 | | | | | |
| N = 225 | | Intercept | −11.044 | — | — | — |
| | | Age | 0.071 | 1.074 | 1.021 | 1.130 |
| | | Weight | 0.047 | 1.048 | 1.016 | 1.082 |
| | AUROC = | Glycemia | 0.429 | 1.536 | 1.120 | 2.106 |
| | 0.941 | | | | | |
| | (0.927) | AST | 0.066 | 1.069 | 1.041 | 1.097 |
| | | ALT | −0.024 | 0.976 | 0.960 | 0.993 |
| | | Ferritin | 0.0009 | 1.001 | 1.000 | 1.002 |
| | | Platelets | −0.0103 | 0.990 | 0.982 | 0.998 |
| | Significant | | | | | |
| | fibrosis | | | | 95% CI | 95% CI |
| | NASH-CRN | | β | | inf | sup |
| 2a | F ≧ 2 | | coeff | Exp (β) | Exp (β) | Exp (β) |
| N = 221 | | Intercept | −6.269 | — | — | — |
| | | Age | 0.033 | 1.034 | 0.996 | 1.072 |
| | | Weight | 0.053 | 1.055 | 1.024 | 1.086 |
| | AUROC = | Glycemia | 0.388 | 1.474 | 1.013 | 2.146 |
| | 0.884 | | | | | |
| | (0.867) | AST | 0.059 | 1.060 | 1.033 | 1.088 |
| | | ALT | −0.014 | 0.986 | 0.973 | 0.998 |
| | | Ferritin | 0.001 | 1.001 | 1.000 | 1.002 |
| | | Prothrombin | −0.046 | 0.955 | 0.916 | 0.996 |
| | | index | | | | |
| | | | | | 95% CI | 95% CI |
| | Area of | | β | | inf | sup |
| 3a | fibrosis | | coeff | | β | β |
| N = 202 | | Intercept | 11.597 | | 5.724 | 17.470 |
| | | Glycemia | 0.441 | | 0.161 | 0.721 |
| | aR² = 0.520 | AST | 0.049 | | 0.023 | 0.076 |
| | (0.503) | ALT | −0.021 | | −0.037 | −0.005 |
| | | Platelets | −0.008 | | −0.016 | 0.001 |
| | | Prothrombin | −0.089 | | −0.151 | −0.027 |
| | | index | | | | |
| | | Hyaluronic | 0.021 | | 0.015 | 0.027 |
| | | acid | | | | |
| | Fractal | | | | 95% CI | 95% CI |
| | dimension | | β | | inf | sup |
| 4a | of fibrosis | | coeff | | β | β |
| N = 202 | | Intercept | 0.920 | | 0.636 | 1.203 |
| | | Age | 0.003 | | 0.001 | 0.005 |
| | aR² = 0.507 | Weight | 0.002 | | 0.0003 | 0.003 |
| | (0.495) | Glycemia | 0.025 | | 0.014 | 0.035 |
| | | AST | 0.001 | | 0.0007 | 0.002 |
| | | Prothrombin | −0.004 | | −0.006 | −0.001 |
| | | index | | | | |
| | | Hyaluronic | 0.001 | | 0.0003 | 0.0008 |
| | | acid | | | | |
| | Significant | | | | 95% CI | 95% CI |
| | steatosis | | β | | inf | sup |
| 5a | (rAOS ≧ 3%) | | coeff | Exp (β) | Exp (β) | Exp (β) |
| N = 151 | | Intercept | −5.090 | — | — | — |
| | | Age | −0.065 | 0.937 | 0.880 | 0.998 |
| | | Sex (M: 0, | 1.422 | 4.145 | 0.905 | 18.971 |
| | | F: 1) | | | | |
| | AUROC = | Triglycerides | 0.956 | 2.602 | 1.203 | 5.624 |
| | 0.866 | | | | | |
| | | Glycemia | 0.479 | 1.614 | 0.974 | 2.675 |
| | | AST/ALT | −2.588 | 0.075 | 0.010 | 0.559 |
| | | Haemoglobin | 0.238 | 1.269 | 0.765 | 2.105 |
| | | Score 1a | −8.416 | <0.001 | <0.001 | 0.071 |
| | | Score 2a | 13.450 | >999.99 | >999.99 | >999.99 |
| | Significant | | | | | |
| | steatosis | | | | 95% CI | 95% CI |
| | NASH-CRN | | β | | inf | sup |
| 6a | grade ≧ 1 | | coeff | Exp (β) | Exp (β) | Exp (β) |
| N = 162 | | Intercept | −2.5933 | — | — | — |
| | | Weight | −0.1066 | 0.899 | 0.846 | 0.956 |
| | | BMI | 0.3864 | 1.472 | 1.189 | 1.822 |
| | AUROC = | Glycemia | 1.2142 | 3.368 | 1.738 | 6.525 |
| | 0.898 | | | | | |
| | | Triglycerides | 0.8002 | 2.226 | 1.141 | 4.344 |
| | | AST | 0.0324 | 1.033 | 1.007 | 1.060 |
| | | AST/ALT | −2.4952 | 0.082 | 0.008 | 0.803 |
| | | Haemoglobin | 0.3510 | 1.421 | 0.899 | 2.246 |
| | | Score 1a | −9.6939 | <0.001 | <0.001 | 0.010 |
| | | Score 2a | 14.8116 | >999.99 | >999.99 | >999.99 |
| | | Score 4a | −14.2847 | <0.001 | <0.001 | 0.674 |
| | Area of | | | | 95% CI | 95% CI |
| | steatosis | | β | | inf | sup |
| 7a | (log) | | coeff | | β | β |
| N = 151 | | Intercept | −2.28009 | | −3.64226 | −0.91793 |
| | | Weight | −0.01128 | | −0.02990 | 0.00735 |
| | | BMI | 0.10169 | | 0.03838 | 0.16500 |
| | aR² = 0.310 | Glycemia | 0.18799 | | 0.05920 | 0.31679 |
| | | Triglycerides | 0.20372 | | 0.04126 | 0.36617 |
| | | AST | 0.00922 | | 0.00222 | 0.01621 |
| | | Score 1a | −3.24488 | | −4.55881 | −1.93095 |
| | | Score 2a | 2.66894 | | 1.34807 | 3.98981 |
| | Fractal | | | | 95% CI | 95% CI |
| | dimension | | β | | inf | sup |
| 8a | of steatosis | | coeff | | β | β |
| N = 151 | | Intercept | 0.95361 | | 0.70956 | 1.19766 |
| | | Weight | −0.00238 | | −0.00529 | 0.00054241 |
| | | BMI | 0.01580 | | 0.00592 | 0.02568 |
| | | Glycemia | 0.02984 | | 0.00932 | 0.05037 |
| | aR² = 0.350 | Triglycerides | 0.03147 | | 0.00637 | 0.05656 |
| | | AST | 0.00438 | | 0.00144 | 0.00733 |
| | | AST/ALT | −0.18234 | | −0.31060 | −0.05407 |
| | | AST.ALT | −0.00002 | | −0.00003124 | −0.00000319 |
| | | Score 1a | −0.40669 | | −0.61323 | −0.20016 |
| | | Score 2a | 0.43128 | | 0.21917 | 0.64338 |
| | | | | | 95% CI | 95% CI |
| | NASH | | β | | inf | sup |
| 9a | (NAS ≧ 3) | | coeff | Exp (β) | Exp (β) | Exp (β) |
| N = 162 | | Intercept | −20.2665 | — | — | — |
| | | AST | 0.1253 | 1.133 | 1.048 | 1.226 |
| | | AST.ALT | −0.00042 | 1.000 | 0.999 | 1.000 |
| | AUROC = | Ferritin | 0.00275 | 1.003 | 1.001 | 1.005 |
| | 0.899 | | | | | |
| | | Score 2a | −4.3346 | 0.013 | <0.001 | 0.285 |
| | | Score 5a | 4.0033 | 54.779 | 1.932 | >999.99 |
| | | Score 8a | 9.4028 | >999.99 | 3.123 | >999.99 |

Diagnostic performance was expressed by either AUROC for score obtained by binary logistic regression or ₐR² for score obtained by multiple linear regression. In these models, the prediction was the main objective so that conditions of independent variables were less stringent than in an explanatory model.
AST: aspartate aminotransferase
ALT: alanine aminotransferase
BMI: body mass index

TABLE 3
Multivariate analyses showing variables independently
associated with different diagnostic targets.

| | | Independent | | |
|---|---|---|---|---|
| Diagnostic target | | variables | Score | Performance |
| Fibrosis: | | | | |
| Significant | Metavir | Weight, age, | #1B | AUROC = 0.932 |
| fibrosis | F ≧ 2 | glycemia, AST, | | (0.918) |
| | | ALT, ferritin, | | |
| | | platelets a | | |
| | NASH-CRN | Weight, glycemia, | #2B | AUROC = 0.866 |
| | F ≧ 2 | AST, ALT, | | (0.856) |
| | | prothrombin | | |
| | | index | | |
| Area of fibrosis | | Hyaluronic acid, | #3B | aR2 = 0.530 |
| | | glycemia, AST, | | (0.518) |
| | | ALT, platelets, | | |
| | | prothrombin | | |
| | | index | | |
| Fractal dimension of | | Hyaluronic acid, | #4B | aR2 = 0.529 |
| fibrosis | | glycemia, | | (0.517) |
| | | AST/ALT, weight, | | |
| | | platelets | | |
| Steatosis: | | | | |
| Significant | rAOS ≧ 3% | BMI, glycemia, | #5B | AUROC = 0.797 |
| steatosis | | triglycerides | | (0.782) |
| | NASH-CRN | BMI, glycemia, | #6B | AUROC = 0.831 |
| | grade ≧ 1 | triglycerides, | | (0.815) |
| | | ferritin | | |
| Area of steatosis | | BMI, glycemia, | #7B | aR2 = 0.213 |
| (log) | | triglycerides, | | (0.198) |
| | | ferritin | | |
| Fractal dimension of | | BMI, glycemia, | #8B | aR2 = 0.247 |
| steatosis | | triglycerides, | | (0.230) |
| | | ferritin | | |
| NASH b: | | | | |
| NAS ≧ 3 | | BMI, AST, | #9B | AUROC = 0.846 |
| | | ferritin | | (0.836) |
| NAS ≧ 5 | | Ferritin | #10B | AUROC = 0.709 |
| | | | | (0.708) |

| | | | |
|---|---|---|---|
| NAS 0-2/3-4/5-7 | BMI, AST, | #11B | AUROC = 0.815 |
| | ferritin | | |

Diagnostic performance was expressed by either AUROC for score obtained by binary/ordinal logistic regression or ₐR² for score obtained by multiple linear regression. Figures into brackets denote AUROC corrected with optimism bias. In these models, the explanation was the main objective so that conditions of independent variables were more stringent than in a predictive model. Variables were selected by the stepwise bootstrap method.
AST: aspartate aminotransferase
ALT: alanine aminotransferase
BMI: body mass index
a The original model determined by conventional binary logistic regression
b According to 3 definitions based on NAS

TABLE 3bis
Multivariate analyses showing variables independently
associated with different diagnostic targets.

| | Diagnostic | | β | | 95% CI | 95% CI |
|---|---|---|---|---|---|---|
| Score | target | Variables | coeff | Exp (β) | inf | sup |
| | Significant | | | | | |
| | fibrosis | | | | | |
| | Metavir | | | | | |
| 1b | F ≧ 2 | | | | Exp (β) | Exp (β) |
| | | Intercept | −6.9269 | — | — | — |
| | | Age | 0.0671 | 1.069 | 1.016 | 1.126 |
| | | Glycemia | 0.4239 | 1.528 | 1.125 | 2.076 |
| | | AST | 0.0585 | 1.060 | 1.034 | 1.087 |
| | | ALT | −0.0184 | 0.982 | 0.968 | 0.996 |
| | | Ferritin | 0.00112 | 1.001 | 1.000 | 1.002 |
| | | Platelets | −0.0105 | 0.990 | 0.982 | 0.997 |
| | Significant | | | | | |
| | fibrosis | | | | | |
| | NASH-CRN | | | | | |
| 2b | F ≧ 2 | | | | Exp (β) | Exp (β) |
| | | Intercept | −3.6323 | — | — | — |
| | | Weight | 0.0539 | 1.055 | 1.026 | 1.086 |
| | | Glycemia | 0.5166 | 1.676 | 1.131 | 2.485 |
| | | AST | 0.0695 | 1.072 | 1.044 | 1.101 |
| | | ALT | −0.0189 | 0.981 | 0.969 | 0.994 |
| | | Prothrombin | −0.0594 | 0.942 | 0.905 | 0.981 |
| | | index | | | | |
| | Area of | | | | | |
| 3b | fibrosis | | | | β | β |
| | | Intercept | 11.59720 | | 5.72423 | 17.47017 |
| | | Glycemia | 0.44087 | | 0.16118 | 0.72055 |
| | | AST | 0.04949 | | 0.02291 | 0.07606 |
| | | ALT | −0.02083 | | −0.03661 | −0.00505 |
| | | Platelets | −0.00752 | | −0.01641 | 0.00138 |
| | | Prothrombin | −0.08903 | | −0.15085 | −0.02721 |
| | | index | | | | |
| | | Hyaluronic | 0.02122 | | 0.01509 | 0.02736 |
| | | acid | | | | |
| | Fractal | | | | | |
| | dimension | | | | | |
| 4b | of fibrosis | | | | β | β |
| | | Intercept | 0.74800 | | 0.59622 | 0.89978 |
| | | Weight | 0.00247 | | 0.00120 | 0.00374 |
| | | Glycemia | 0.02634 | | 0.01630 | 0.03637 |
| | | AST/ALT | 0.19838 | | 0.12788 | 0.26888 |
| | | Platelets | −0.0005524 | | −0.0008494 | −0.0002553 |
| | | Hyaluronic | 0.00035650 | | 0.00011211 | 0.00060090 |
| | | acid | | | | |
| | Significant | | | | | |
| | steatosis | | | | | |
| 5b | (rAOS ≧ 3%) | | | | Exp (β) | Exp (β) |
| | | Intercept | −10.8186 | — | — | — |
| | | BMI | 0.2667 | 1.306 | 1.136 | 1.500 |
| | | Glycemia | 0.5154 | 1.674 | 1.047 | 2.678 |
| | | Triglycerides | 0.9728 | 2.645 | 1.388 | 5.042 |
| | Significant | | | | | |
| | steatosis | | | | | |
| | NASH-CRN | | | | | |
| 6b | grade ≧ 1 | | | | Exp (β) | Exp (β) |
| | | Intercept | −11.1031 | — | — | — |
| | | BMI | 0.2124 | 1.237 | 1.108 | 1.380 |
| | | Glycemia | 0.6451 | 1.906 | 1.180 | 3.080 |
| | | Triglycerides | 0.7650 | 2.149 | 1.261 | 3.663 |
| | | Ferritin | 0.00149 | 1.001 | 1.000 | 1.003 |
| | Area of | | | | | |
| | steatosis | | | | | |
| 7b | (log) | | | | β | β |
| | | Intercept | −1.78517 | | −2.98336 | −0.58698 |
| | | BMI | 0.08947 | | 0.05079 | 0.12815 |
| | | Glycemia | 0.09542 | | −0.02158 | 0.21241 |
| | | Triglycerides | 0.22814 | | 0.05665 | 0.39963 |
| | | Ferritin | 0.0002852 | | −0.0001074 | 0.0006778 |
| | Fractal | | | | | |
| | dimension | | | | | |
| 8b | of steatosis | | | | β | β |
| | | Intercept | 0.91010 | | 0.72363 | 1.09658 |
| | | BMI | 0.01502 | | 0.00900 | 0.02103 |
| | | Glycemia | 0.01625 | | −0.00195 | 0.03446 |
| | | Triglycerides | 0.03534 | | 0.00865 | 0.06203 |
| | | Ferritin | 0.0000576 | | −0.0000035 | 0.0001187 |
| | NASH | | | | | |
| 9b | (NAS ≧ 3) | | | | Exp (β) | Exp (β) |
| | | Intercept | −7.5364 | — | — | — |
| | | BMI | 0.1596 | 1.173 | 1.059 | 1.300 |
| | | AST | 0.0479 | 1.049 | 1.024 | 1.075 |
| | | Ferritin | 0.00256 | 1.003 | 1.001 | 1.004 |
| | NASH | | | | | |
| 10b | (NAS ≧ 5) | | | | | |
| | | Intercept | −2.4712 | — | — | — |
| | | Ferritin | 0.00148 | 1.001 | 1.001 | 1.002 |
| | NASH | | | | | |
| | (NAS 0-2/ | | | | | |
| 11b | 3-4/5-7) | | | | | |
| | | Intercept3 | −7.3898 | — | — | — |
| | | Intercept2 | −5.3943 | — | — | — |
| | | BMI | 0.1258 | 1.134 | 1.048 | 1.228 |
| | | AST | 0.0255 | 1.026 | 1.010 | 1.042 |
| | | Ferritin | 0.00158 | 1.002 | 1.001 | 1.003 |

Diagnostic performance was expressed by either AUROC for score obtained by binary/ordinal logistic regression or ₐR² for score obtained by multiple linear regression. Figures into brackets denote AUROC corrected from optimism bias. In these models, the explanation was the main objective so that condition of non-collinearity between independent variables was more stringent than in a predictive model. Variables were selected by a bootstrap method (stepwise regression analysis on 1,000 bootstrap samples).

### Area and Fractal Dimension of Fibrosis

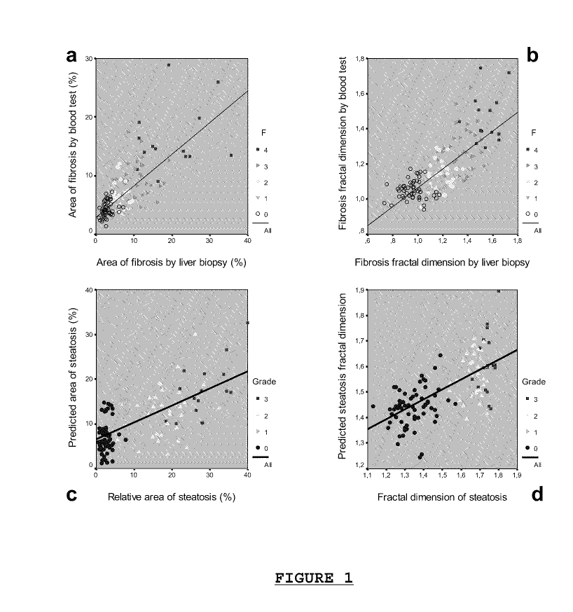

By multiple linear regression, the highest accuracy was obtained with a combination of the independent predictors of AOF (see tables 2 and 3). Thus, the correlation between AOF predicted by blood test and measured by liver biopsy was: rₚ=0.731 (p<10⁻³) (FIG. 1a). The highest accuracy for fibrosis FD was obtained with a combination of the independent predictors (see tables 2 and 3). Thus, the correlation between fibrosis FD predicted by blood test and measured by liver biopsy was: rₚ=0.734 (p<10⁻³) (FIG. 1b).

### Diagnosis of Liver Steatosis

### Significant Steatosis

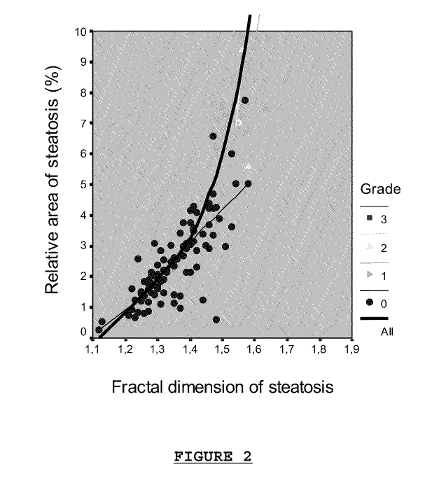

Diagnostic performance—Significant steatosis was defined in two ways. It was first defined by rAOS ≧3% since the plot area vs FD showed an inflexion at rAOS≈3% and FD≈1.4 (FIG. 2). The second definition was based on NASH-CRN grade ≧1. The prevalence of steatosis was significantly higher with rAOS ≧3%:68.8% than with NASH-CRN grade ≧1: 54.7% (p<10⁻³ by McNemar test).

Using binary logistic regression, significant steatosis defined by rAOS ≧3% was diagnosed by the independent variablesas shown in tables 2 and 3. Diagnostic performance of this model was: AUROC: 0.797±0.027 (95% CI: 0.804-0.911), overall accuracy: 77.4%.

By using NASH-CRN grade ≧1 as diagnostic target, the best score is shown in tables 2 and 3.

### Steatosis Amount

Using multiple linear regression, the rAOS could be estimated, with a moderate accuracy (ₐR²=0.213), by a score including blood and clinical variables as shown in tables 2 and 3. Thus, the correlation between rAOS predicted by blood test and measured by liver biopsy was: rₚ=0.647 (p<10⁻³) (FIG. 1c).

### Fractal Dimension of Steatosis

Using multiple linear regression, the steatosis FD could be estimated, with a moderate accuracy (ₐR²=0.247), by a score including blood and clinical variablesas shown in tables 2 and 3. Thus, the correlation between steatosis FD predicted by blood test and measured by liver biopsy was: rₚ=0.621 (p<10⁻³) (FIG. 1d).

### Diagnosis of NASH

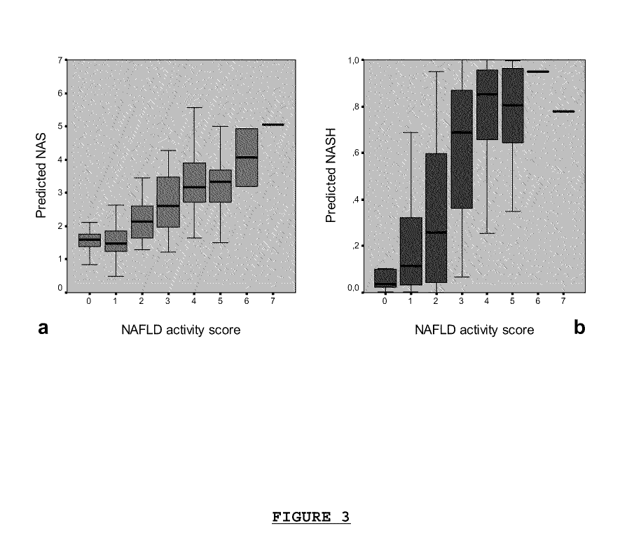

NASH was independently predicted as shown in tables 2 and 3 with corresponding AUROC. The correlation between NASH predicted by blood test and NAS measured by liver biopsy was: rₛ=0.726 (p<10⁻³) (FIG. 3b).

### Relationship Between Lesions

In order to validate the blood tests, we evaluated the ability of those tests to reflect the relationships between lesions.

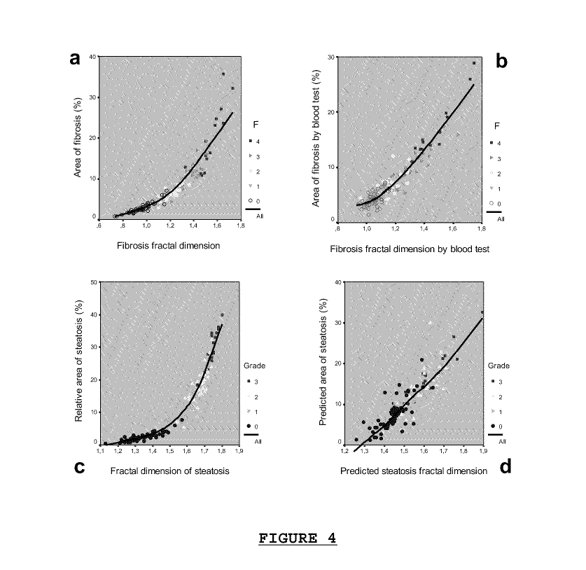

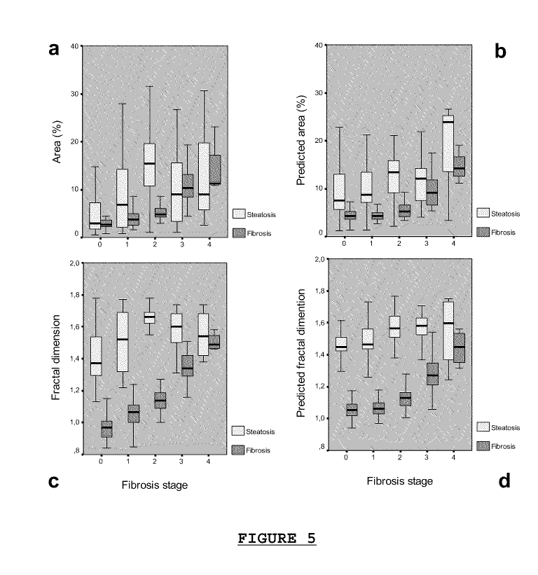

Area and FD—The exponential relationship between histological AOF and FD (rₛ=0.971, p<10⁻³) was well reflected by the relationship between respective blood tests (rₛ=0.861, p<10⁻³) (FIG. 4a and b). The exponential relationship between histological rAOS and steatosis FD (rₛ=0.976, p<10⁻³) was well reflected by the relationship between respective blood tests (rₛ=0.886, p<10⁻³) (FIG. 4c and d).
Fibrosis and steatosis—NASH-CRN fibrosis stage is considered here as a chronological reference since it is assumed to increase as a function of time. AOS peaked in NASH-CRN fibrosis stage 2 with a quadratic curve whereas AOF almost linearly increased as a function of fibrosis stage (FIG. 5a). These relationships were well reflected by respective blood tests except for AOS in F4 but there were a few patients (FIG. 5b). The same curves were observed with FD of fibrosis or steatosis (FIG. 5c) whereas they were well reflected by respective blood tests (FIG. 5d).

### MRI Studies

21 patients with steatohepatitis were included, along with two controls without steatohepatitis.

Liver biopsy was performed in order to diagnose alcoholic or non-alcoholic steatohepatitis.

Steatosis degree was evaluated by an expert using steatosis grading
S0 (0% of hepatocytes),
S1 (less than 10%),
S2 (between 10% and 30%),
S3 (between 30% and 50%) and
S4 (more than 50%).

Steatosis area (AOS) (in percentage) was computed by our automatic image analysis method, previously described in the present application, which measures the area of lipid vacuoles presented in hepatocytes.

Quantification methods by MRI, already described in the literature, were implemented, namely: Hussain, Fast Spin Echo with and without fat saturation (FSE), 2-point Dixon and 3-point Dixon.

An additional method based on multi-echo gradient-echo MRI (MFGRE) was developed, as previously described in the present application, and applied.

Correlations were evaluated by Spearman correlation coefficient (Rs) and intraclass correlation coefficient (Ric).

### Results

The distribution of our population by steatosis grading was:
S0=2 patients,
S1=4 patients,
S2=4 patients,
S3=7 patients and
S4=6 patients.

### Correlation Between Steatosis Grading and AOS:

AOS was correlated with steatosis grades (Rs=0.82 and p<0.001).

AOS was significantly different between all steatosis grades (p<0.001), e.g. between S0 to S2 grades (less than 30%) and S3 to S4 grades (more than 30%), respectively: 4.2±2.4 versus 16.4±8.9 (p<0.001).

### Correlation Between MRI Quantification Methods and Steatosis Grading:

Intraclass correlation between steatosis grades and MRI quantification methods was Rs=0.66 for MFGRE, Rs=0.69 for Hussain, Rs=0.80 for FSE; Rs=0.68 for 2-point Dixon and Rs=0.73 for 3-point Dixon (p<0.001 for all correlations).

### Correlation Between MRI Quantification and AOS:

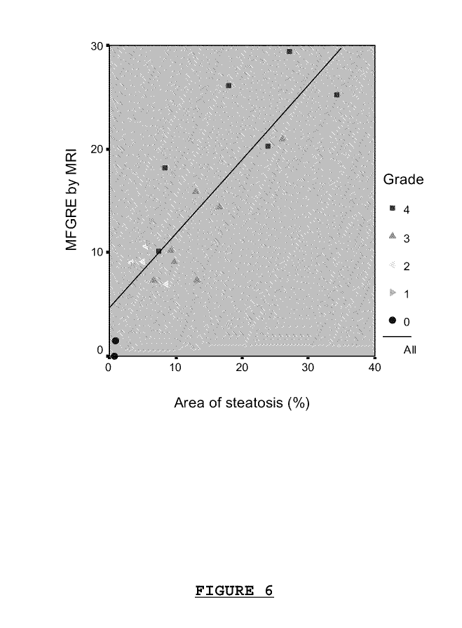

Steatosis quantification by MFGRE method correlated best to AOS (Rs=0.77 and Ric=0.85) (see FIG. 6).

Correlation coefficients obtained by other methods were, respectively: 0.71 and 0.57 for Hussain, 0.80 and 0.61 for FSE; 0.71 and 0.55 for 2-point Dixon; 0.61 and 0.28 for 3-point Dixon.

In multiple linear regression, MFGRE was the only MRI quantification method independently linked to AOS.

Although the present invention has been described hereinabove by way of preferred embodiments thereof, it can be modified, without departing from the spirit and nature of the subject invention as defined in the appended claims.

1. An in-vitro non-invasive method for quantifying the lesions of the liver of the patient comprising addressing a diagnostic target, i.e. fibrosis, steatosis and/or NASH and measuring at least one marker selected from the group consisting of biomarkers and possibly clinical markers and possibly scores, the biomarkers being selected from the group consisting of glycemia, AST (aspartate aminotransferase), ALT (alanine aminotransferase), AST/ALT, AST.ALT, ferritin, platelets, prothrombin index, hyaluronic acid, haemoglobin, triglycerides; the clinical biomarker being selected from the group consisting of weight, body mass index, sex and age, hip perimeter, abdominal perimeter and the ratio thereof, such as for example hip perimeter/abdominal perimeter; and the scores being selected from the group consisting of score of fibrosis, area of fibrosis, fractal dimension of fibrosis, score of steatosis, area of steatosis, fractal dimension of steatosis and combining said measures in a mathematical function combining said measures through a logistic function including said markers in order to obtain an end value.

2. The method according to claim 1, wherein addressing a diagnostic target is performed by carrying out a score, an area or a fractal dimension of fibrosis in a blood sample of the patient, and/or carrying out a score, an area or a fractal dimension of steatosis in said patient and/or carrying out a score of steato-hepatitis (NASH) in said patient.

3. The method according to claim 1, wherein 2,3,4,5,6,7,8,9 markers are measured and combined.

4. The method according to claim 1, wherein the lesion is due or related to liver impairment, liver steatosis, non-alcoholic fatty liver disease (NAFLD), steatohepatitis, or non-alcoholic steatohepatitis (NASH).

5. The method according to claim 1, wherein the target is fibrosis, and a score, area and/or fractal dimension of fibrosis is performed, and at least 5, preferably 5,6,7,8 markers, are measured, said markers being selected from the group consisting of glycemia, AST, ALT, AST/ALT, ferritine, platelets, prothrombin index, hyaluronic acid, haemoglobin, triglycerides, weight, body mass index, sex.

6. The method according to claim 1, wherein the target is steatosis and a score, area and/or fractal dimension of steatosis is performed, and at least 5 markers are measured, said markers selected from the group consisting of glycemia, AST, ALT, AST/ALT ferritine, platelets, prothrombin index, hyaluronic acid, haemoglobin, triglycerides, weight, body mass index, sex and age, and optionally, a score, area or fractal dimension of fibrosis.

7. The method according to claim 1, wherein the target is inflammation, and at least one marker is measured, said markers selected from the group consisting of AST, ferritine, AST.ALT, body mass index, and optionally, at least one score selected from the score of fibrosis, the score of significant steatosis, and the fractal dimension of steatosis score.

8. The method according to claim 1, wherein the diagnostic target is fibrosis, and
a score is carried out wherein the biomarkers are selected from the group consisting of weight, age, glycemia, AST, ALT, ferritin and platelets or from the group consisting of weight, age, glycemia, AST, ALT, ferritin and prothrombin index; or
an area is carried out wherein the biomarkers are selected from the group consisting of hyaluronic acid, glycemia, AST, ALT, platelets, and prothrombin index; or
a fractal dimension is carried out wherein the biomarkers are selected from the group consisting of weight, Hyaluronic acid, glycemia, AST, age, and prothrombin index.

9. The method according to claim 1, wherein the diagnostic target is steatosis and
a score is carried out wherein the biomarkers are selected from the group consisting of glycemia, AST/ALT, triglycerides, haemoglobin, age, and sex and optionally at least one fibrosis score; or from the group consisting of glycemia, AST, triglycerides, AST/ALT, haemoglobin, BMI, and weight and optionally fibrosis scores and/or fractal dimension of fibrosis score; or from the group consisting of BMI, glycemia, and triglycerides, or from the group consisting of BMI, glycemia, triglycerides and ferritin; or
an area is carried out wherein the biomarkers are selected from the group consisting of glycemia, AST, triglycerides, BMI, weight and optionally at least one fibrosis score; or from the group consisting of BMI, glycemia, triglycerides and ferritin; or
a fractal dimension is carried out wherein the biomarkers are selected from the group consisting of glycemia, AST, ALT, triglycerides, BMI, weight and optionally at least one fibrosis score; or from the group consisting of BMI, glycemia, triglycerides and ferritin.

10. The method according to claim 1, wherein the diagnostic target is NASH, and the biomarkers are selected from the group consisting of AST, ferritin, and AST.ALAT and optionally fibrosis score and/or steatosis score and/or NASH score; or from the group consisting of BMI, AST, and ferritin; or ferritin alone

11. An non-invasive method for quantifying the lesions of the liver of the patient, comprising performing a multi-echo gradient-echo MRI called MFGRE on whole or part of the liver of the patient, measuring the liver fat content on the resulting MRI signal and comparing said liver fat content on the resulting MRI signal to the area of lipid vacuoles of the reference image.

12. A method according to claim 1, wherein the target is steatosis, comprising:
a) measuring the fat content on a MRI signal of the liver of a patient by a non-invasive method for quantifying the lesions of the liver of the patient, comprising performing a multi-echo gradient-echo MRI called MFGRE on whole or part of the liver of the patient, measuring the liver fat content on the resulting MRI signal and comparing said liver fat content on the resulting MRI signal to the area of lipid vacuoles of the reference image;
b) performing a score, area and/or fractal dimension of steatosis, and at least 5 markers are measured, said markers selected from the group consisting of glycemia, AST, ALT, AST/ALT ferritine, platelets, prothrombin index, hyaluronic acid, haemoglobin, triglycerides, weight, body mass index, sex and age, and optionally, a score, area or fractal dimension of fibrosis, measuring the level of at least one marker and/or Fibrosis Score;
c) combining the measured fat content index obtained in step (a) and the measured levels, score and/or clinical markers obtained in step (b) in a suitable mathematical function.

13. The method according to claim 2, wherein 2,3,4,5,6,7,8,9 markers are measured and combined.

14. The method according to claim 2, wherein the lesion is due or related to liver impairment, liver steatosis, non-alcoholic fatty liver disease (NAFLD), steatohepatitis, or non-alcoholic steatohepatitis (NASH).

15. The method according to claim 3, wherein the lesion is due or related to liver impairment, liver steatosis, non-alcoholic fatty liver disease (NAFLD), steatohepatitis, or non-alcoholic steatohepatitis (NASH).

16. The method according to claim 2, wherein the target is fibrosis, and a score, area and/or fractal dimension of fibrosis is performed, and at least 5, preferably 5,6,7,8 markers, are measured, said markers being selected from the group consisting of glycemia, AST, ALT, AST/ALT, ferritine, platelets, prothrombin index, hyaluronic acid, haemoglobin, triglycerides, weight, body mass index, sex.

17. The method according to claim 3, wherein the target is fibrosis, and a score, area and/or fractal dimension of fibrosis is performed, and at least 5, preferably 5,6,7,8 markers, are measured, said markers being selected from the group consisting of glycemia, AST, ALT, AST/ALT, ferritine, platelets, prothrombin index, hyaluronic acid, haemoglobin, triglycerides, weight, body mass index, sex.

18. The method according to claim 4, wherein the target is fibrosis, and a score, area and/or fractal dimension of fibrosis is performed, and at least 5, preferably 5,6,7,8 markers, are measured, said markers being selected from the group consisting of glycemia, AST, ALT, AST/ALT, ferritine, platelets, prothrombin index, hyaluronic acid, haemoglobin, triglycerides, weight, body mass index, sex.

19. The method according to claim 2, wherein the target is steatosis and a score, area and/or fractal dimension of steatosis is performed, and at least 5 markers are measured, said markers selected from the group consisting of glycemia, AST, ALT, AST/ALT ferritine, platelets, prothrombin index, hyaluronic acid, haemoglobin, triglycerides, weight, body mass index, sex and age, and optionally, a score, area or fractal dimension of fibrosis.

20. The method according to claim 3, wherein the target is steatosis and a score, area and/or fractal dimension of steatosis is performed, and at least 5 markers are measured, said markers selected from the group consisting of glycemia, AST, ALT, AST/ALT ferritine, platelets, prothrombin index, hyaluronic acid, haemoglobin, triglycerides, weight, body mass index, sex and age, and optionally, a score, area or fractal dimension of fibrosis.
